(19) United States
(12) Patent Application Publication
(10) Pub. No.: US 2012/0238533 A1
(43) Pub. Date: Sep. 20, 2012

(54) (Heteroarylmethyl) Thiohydantoins as anticancer drugs

(75) Inventors: Arwed Cleve, Berlin (DE); Ulrich Lücking, Berlin (DE); Stefan Bäurle, Berlin (DE); Martin Fritsch, Berlin (DE); Jens Schröder, Berlin (DE); Bernd-Wolfgang Igl, Berlin (DE)

(73) Assignee: BAYER PHARMA AKTIENGESELLSCHAFT, Berlin (DE)

(21) Appl. No.: 13/395,087

(22) PCT Filed: Sep. 3, 2010
(86) PCT No.: PCT/EP2010/062964

§ 371 Date: May 18, 2012

(30) Foreign Application Priority Data
Sep. 11, 2009 (EP) 09075421.9

(51) Int. Cl. A61K 31/4166; A61K 31/4439; A61K 31/57; A61K 31/167; A61K 31/56; A61K 31/407; A61P 35/00; A61K 31/565; A61K 31/366; A61K 31/277; A61K 31/4704

(52) U.S. Cl. 514/178; 514/522; 514/391; 514/341; 514/629; 514/312; 514/411; 514/453; 514/169; 514/410

(57) ABSTRACT

The invention refers to the use of androgen receptor antagonists for the treatment and/or prevention of fibroids, also known as uterine leiomyoma, leiomyomata. Particularly, the invention refers to the use of an androgen receptor antagonist being any one of the compounds according to the following list: cyproterone acetate, oxendolone, chlormadinone acetate, spironolactone, osaterone acetate, dienogest, flutamide, hydroxyflutamide, nilutamide, bicalutamide, RU 58841, LGD-2226, MDV3100, BMS-641988, BMS-779333, or 4-(3-{[6-(2-hydroxy-2-methylpropoxy)pyridin-3-yl]methyl}-4,4-dimethyl-5-oxo-2-thioxoimidazolidin-1-yl)-2-(trifluoromethyl)benzonitrile (thioxoimidazolidine derivative) for the treatment of fibroids.

### TECHNICAL FIELD

The present invention is directed to the use of androgen receptor antagonists for curing and/or preventing uterine fibroids.

A further aspect of the present invention refers to steroidal and non-steroidal androgen receptor antagonists for curing and/or preventing.

In particular, an androgen receptor antagonist for the treatment of fibroids can be for example any of, but not limited to, the following compounds:

| | |
|---|---|
| 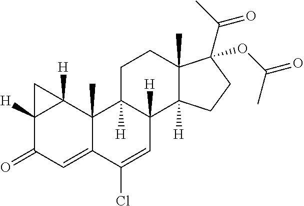 | I |
| 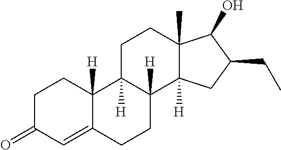 | II |
| 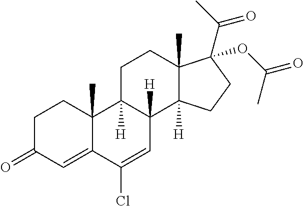 | III |
| 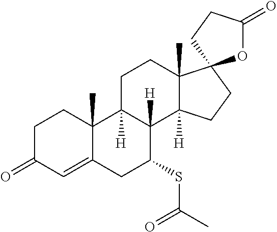 | IV |
| 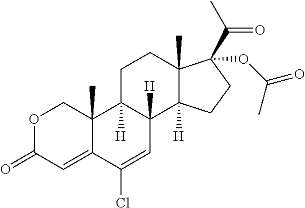 | V |
| 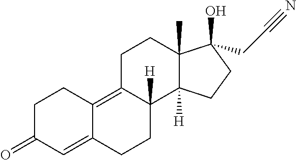 | VI |
| 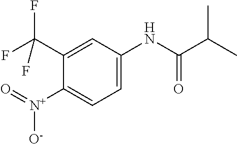 | VII |
| 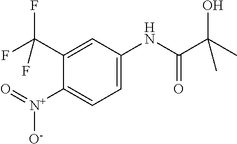 | VIII |
| 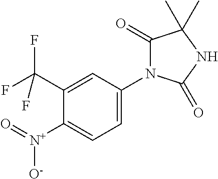 | IX |
| 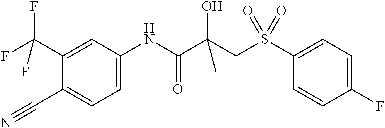 | X |
| 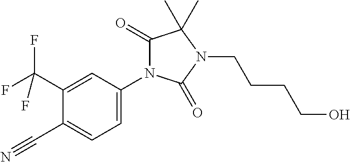 | XI |
| 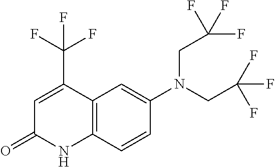 | XII |
| 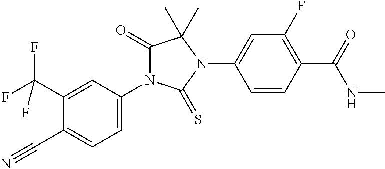 | XIII |
| 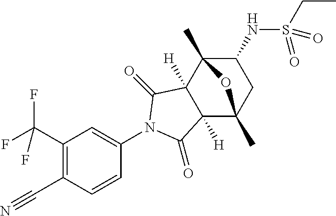 | XIV |
| 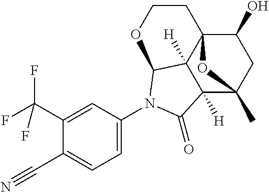 | XV |
| 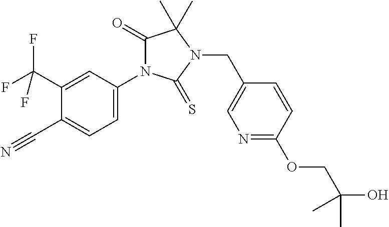 | XVI |

| | | |
|---|---|---|
| I | Cyproterone | (1R,3aS,3bR,7aR,8aS,8cS,10aS)-1-acetyl-5- |
| | acetate | chloro-1-hydroxy-8b,10a-dimethyl-2,3,3a, |
| | | 3b,7a,8,8a,8b,8c,9,10,10a-dodecahydro- |
| | | cyclopenta[a]cyclopropa[g]phenanthren- |
| | | 7(1H)-one |
| II | Oxendolone | 16β-ethyl-17β-hydroxyestr-4-en-3-one |
| III | Chlormadinone | 6-chloro-3,20-dioxopregna-4,6-dien-17-yl |
| | acetate | acetyl |
| IV | Spironolactone | S-[(7R,8R,9S,10R,13S,14S,17R)-10,13- |
| | | dimethyl-3,5′-dioxo-1,2,3,4′,5′,6,7,8,9,10,11, |
| | | 12,13,14,15,16-hexadecahydro-3′H-spiro |
| | | [cyclopenta[a]phenanthrene-17,2′-furan]-7- |
| | | yl]ethanethioate |
| V | Osaterone | (4aR,4bS,6aS,7R,9aS,9bR)-7-acetyl-11- |
| | acetate | chloro-4a,6a-dimethyl-2-oxo-2,4,4a,4b,5,6, |
| | | 6a,7,8,9,9a,9b-dodecahydroindeno[4,5-h] |
| | | isochromen-7-yl acetate |
| VI | Dienogest | 17-Hydroxy-3-oxo-19-nor-17α-pregna-4,9- |
| | | diene-21-nitrile |
| VII | Flutamide | 2-methyl-N-[4-nitro-3-(trifluoromethyl) |
| | | phenyl]propanamide |
| VIII | Hydroxy- | 2-hydroxy-2-methyl-N-[4-nitro-3- |
| | flutamide | (trifluoromethyl)phenyl]propanamide |
| IX | Nilutamide | 5,5-dimethyl-3-[4-nitro-3-(trifluoromethyl) |
| | | phenyl]imidazolidine-2,4-dione |
| X | Bicalutamide | N-[4-cyano-3-(trifluoromethyl)phenyl]-3- |
| | | [(4-fluorophenyl)sulfonyl]-2-hydroxy-2- |
| | | methylpropanamide |
| XI | RU 58841 | 4-(4,4-dimethyl-2,5-dioxo-3-(4-hydroxybutyl) |
| | | 1-imidazolidinyl)-2-(trifluoromethyl)- |
| | | benzonitrile |
| XII | LGD-2226 | 6-(bis-2,2,2-trifluoroethyl)amino-4- |
| | | trifluoromethyl-2(1H)-quinoline |
| XIII | MDV3100 | N-methyl-4-[3-(4-cyano-3-trifluoromethyl- |
| | | phenyl)-5,5-dimethyl-4-oxo-2-thioxo- |
| | | imidazolidin-1-yl]-2-fluorobenzamide, |
| | | RD162′ |
| XIV | BMS-641988 | (3aα,4β,5α,7β,7aα)-4-(octahydro-5-ethyl- |
| | | sulfonamido-4,7-dimethyl-1,3-dioxo-4,7- |
| | | epoxy-2H-isoindol-2-yl)-2-(trifluoro- |
| | | methyl)benzonitrile |
| XV | BMS-779333 | 4-((1R,2S,4R,5S,8S,12R)-2-hydroxy-4-methyl |
| | | 6-oxo-9,13-dioxa-7-azatetracyclo |
| | | [6.3.1.1^{1,4}.0^{5,12}]tridec-7-yl)-2- |
| | | (trifluoromethyl)benzonitrile |
| XIV | Thioxo- | 4-(3-{[6-(2-hydroxy-2-methylpropoxy)pyridin- |
| | imidazolidine | 3-yl]methyl}-4,4-dimethyl-5-oxo-2- |
| | derivative | thioxoimidazolidin-1-yl)-2-(trifluoromethyl) |
| | | benzonitrile |

The present invention further refers to a pharmaceutical composition comprising an androgen receptor antagonist, for example any one of the previously mentioned compounds, for curing and/or preventing uterine fibroids in a mammal, particularly a human.

In another aspect, this invention is directed to methods of treating and/or preventing uterine fibroids in a mammal, particularly a human, wherein the method comprises administering to the mammal in need thereof a therapeutically effective amount of an androgen receptor antagonist, particularly any one of the compounds described above.

### BACKGROUND ART

Uterine fibroids, also known as uterine leiomyoma, leiomyomata, or simply fibroids or myoma, are benign tumors of the uterine muscle or myometrium that affect 25-40% of women of reproductive age (Nowak R A. “Fibroids: pathophysiology and current medical treatment” Baillieres Best Pract Res Clin Obstet Gynaecol 1999; 13: 223-238; Walker C L. “Role of hormonal and reproductive factors in the etiology and treatment of uterine leiomyoma”, Recent Prog Horm Res 2002; 57, 277-294)

Fibroids grow under the influence of the steroid hormones estrogen and progestin. It appears that the incidence of fibroids strongly parallels the reproductive changes in progesterone and estrogen over the life span of women, suggesting hormonal regulation. For instance, about 25% of reproductive-aged women report symptoms consistent with fibroids, but the number of women reporting symptoms decreases at the time of menopause (Severino M F, Murray M J, Brandon D D, Clinton G M, Burry K A, Novy M J. “Rapid loss of oestrogen and progesterone receptors in human leiomyoma and myometrial explant cultures”, Mol Hum Reprod 1996; 2: 823-828). Therefore, in parallel with the hormone levels, fibroids generally regress after the onset of menopause.

Fibroids may develop in different locations within the myometrium. Subserosal fibroids are located directly under the serosa of the peritoneum, intramural fibroids are within the myometrium and submucosal fibroids develop below the endometrium and generally do influence the shape of the uterine cavity. Since fibroids generally occur in multiples, and may grow very large, they lead to an irregularly enlarged and usually asymmetrical uterus. Compared to the myometrium, the fibroid itself is very stiff due to the abnormally enlarged deposition of disordered extracellular matrix.

The cells within a fibroid grow in whirls that form ball- or irregular shaped benign tumors varying from 1 mm to over 20 cm in diameter. These smooth muscle tumors might be asymptomatic.

Fibroids are frequently associated with and can be the cause of a variety of symptoms including heavy menstrual flow, bleeding between periods, pain, infertility, pelvic pressure, stress urinary incontinence, and urethral obstruction.

The diagnosis is usually based on the clinical findings of an enlarged, irregularly shaped, firm uterus. Sometimes, the diagnosis is unclear and diagnostic tests are used to delineate the fibroids and rule out other problems. Presently the diagnosis is based on: ultrasound, MRI and CT scanning, laparoscopy, histology.

Due to a lack of effective medical therapies, gynecologic symptoms due to fibroids frequently result in surgical intervention. Fibroids are one of the most common clinical conditions leading to hysterectomy, further alternatives are the myomectomy, a conservative uterus-sparing surgical procedure, and uterine artery embolization. However, these procedures are invasive and expensive and may allow for recurrence of fibroids and symptoms.

Therefore there is still an unmet medical need for effective treatment of uterine fibroids.

The androgen receptor (AR) is a member of the steroid and nuclear receptor superfamily, which do act as transcription factors. Binding of androgens to the AR leads to its stabilization by a conformational change, a subsequent reduced proteolytic susceptibility, and its eventual transport into the nucleus. There, the AR binds to androgen-receptor responsive DNA elements located in the promotor region of specific genes. Binding of the AR-androgen complex to its respective DNA binding element generally leads to an increased activation of the respective gene (D. J. Lamb et. al. Vitam. Horm. 2001, 62, 199-230).

AR is mainly expressed in androgen target tissues, such as the prostate, skeletal muscle, liver, and central nervous system (CNS), with the highest expression level observed in the prostate, adrenal gland, and epididymis as determined by real-time polymerase chain reaction (PCR).

As described above, the AR can be activated by the binding of endogenous androgens, including testosterone and 5α-dihydrotestosterone (5α-DHT). The actions of androgens in the reproductive tissues are known as the androgenic effects, while the nitrogen retaining effects of androgen in muscle and bone are known as the anabolic effects. To date, only one AR gene has been identified in humans.

Anti-androgens or androgen receptor antagonists, by definition, antagonize the actions of testosterone or 5α-DHT by competing for AR binding sites. Such compounds have therapeutic potential in the treatment of prostate cancer, BPH, acne, virilization in women, and male contraception (Wenqing Gao, Casey E. Bohl, and James T. Dalton; “Chemistry and Structural Biology of Androgen Receptor” Chem. Rev. 2005, 105, 3352-3370). In addition, a more differential therapeutic favorable effect in the affected tissue, or differential effects in different tissues might be achieved by the concept of selective androgen receptor modulators (SARMs).

SARMs might displace the physiologically AR activating compounds such as testosterone and 5α-DHT, but do activate transcription in different tissues in different manners and intensities as the former androgens do. On a molecular biology level, it is presumed that SARMs induce conformational changes in the AR different to those induced by testosterone and 5α-DHT. This causes the binding of only a subgroup or of even completely different proteins to the receptor which leads to a tissue-specific modulation of the transcriptional activity and the subsequent signal transduction pathways of the AR (Ramesh Narayanan, Christopher C. Coss, Muralimohan Yepuru, Jeffrey D. Kearbey, Duane D. Miller, and James T. Dalton; “Steroidal Androgens and Nonsteroidal, Tissue-Selective Androgen Receptor Modulator, S-22, Regulate Androgen Receptor Function through Distinct Genomic and Nongenomic Signaling Pathways” Mol. Endocrinol. 2008, 22(11), 2448-2465). On the level of an intact organism, the differential activation of the AR in different tissues might be typically assessed by the Hershberger assay, in which the differential anabolic and androgenic activity of a SARM is evaluated by its growth stimulating activities on the musculus levator ani and seminal vesicles or the prostate, respectively, in comparison to the activity of a reference androgen like testosterone or 5α-DHT (Michael L. Mohler, Casey E. Bohl, Amanda Jones, Christopher C. Coss, Ramesh Narayanan, Yali He, Dong Jin Hwang, James T. Dalton, and Duane D. Miller “Nonsteroidal Selective Androgen Receptor Modulators (SARMs): Dissociating the Anabolic and Androgenic Activities of the Androgen Receptor for Therapeutic Benefit” J. Med. Chem. 2009, 52(11), 3597-3617).

The structural basis of SARMs might be either steroidal or non-steroidal. The activity of steroidal SARMs in the treated mammal might, by purpose, also affect other steroid receptors such as the progesterone receptor and/or mineralocorticoid receptor.

Furthermore, it was reported that patients with multiple leiomyomas tend to carry a longer CAG repeat structure polymorphism in AR exon 1, with the mean CAG repeat number longer in the multicentric multiple cases (24.1) compared to that of the unicentric, multinodular cases (22.2) and those with solitary lesions (23.1; P<0.01). These results indicate that a longer CAG repeat structure confers women greater susceptibility to leiomyoma development in the uterus (Teng X Y et al., “CAG repeats in the androgen receptor gene are shorter in patients with pulmonary, esophageal or bladder carcinoma and longer in women with uterine leiomyoma.”; Oncol Rep. 2010 March; 23(3):811-8). The publication “Uterine myoma in postmenopause: a comparison between two therapeutic schedules of HRT”, F. Polatti et al., Maturitas 37 (2000) 27-32 discusses whether hormone replacement therapy (HRT) can affect the onset of uterine myomas or their growth in postmenopause. Accordingly, it was suggested that likely some therapeutic schedules could influence the myometrial growth differently, due to a more potent stimulation of the uterine receptors. The study reported in said publication evaluated the effects of two different hormonal treatment schedules on the risk of uterine myoma onset or progression, and in particular compared an oral cyclic administration of estradiol valerate (EV) and cyproterone acetate (CA) versus a sequential combination of transdermal E2 and oral medroxyprogesterone acetate on 240 postmenopausal women with and without uterine myomas over two years. The replacement therapy with EV+CA did not affect the appearance of uterine myomas nor caused increased volume of pre-existing myomas. The use of CA alone to inhibit the growth of myomas, or their formation in general, in view of the anti-androgenic activity of the compound is not disclosed in the article.

WO2009/075334 discloses a uterine fibroid cell growth inhibitor which is free from the risk of causing an adverse side effect including ovarian dysfunction and bone loss, and which can be administered over a long period; and a prophylactic or therapeutic agent for uterine fibroid. Said uterine fibroid cell growth inhibitor comprises in particular an aldosterone receptor inhibitor as an active ingredient. Spironolactone is disclosed as active ingredient, however no reference is made to the anti-androgenic activity of said compound in relation to its ability of inhibiting uterine fibroid cell growth.

EP 1029868 (A1) discloses a hysteromyoma remedy containing dienogest or a solvate thereof as the active ingredient. It is reduced in adverse effects, suppressed in the rebound after administration, capable of being used alone or in combination with a GnRH agonist, and capable of administration and pharmaceutical preparation as peroral and percutaneous drugs and suppositories. Dienogest has also an anti-androgen component, however no connection between the activity of dienogest as anti-androgen and the possibility of treating hysteromyoma by means of said activity is disclosed in the above document.

Uterine leiomyoma, as the myometrium of the uterus, do express the androgen receptor on a mRNA (Jiro Fujimoto,* Miki Nishigaki, Masashi Hori, Satoshi Ichigo, Toshiya Itoh and Teruhiko Tamaya, “The Effect of Estrogen and Androgen on Androgen Receptors and mRNA Levels in Uterine Leiomyoma, Myometrium and Endometrium of Human Subjects”, Steroid Biochem. Molec. Biol. 1994, 50(3/4): 137-43) and on a protein level (T. Tamaya, J. Fujimoto and H. Okada “Comparison of Cellular Levels of Steroid Receptors in Uterine Leiomyoma and Myometrium”, Acta Obstet Gynecol Scand 1985, 64: 307-309). Serial histological analysis of Leiomyoma in immunohistochemical studies showed an expression of the androgen receptor in situ (Leitao M M, Soslow R A, Nonaka D, Olshen A B, Aghajanian C, Sabbatini P, Dupont J, Hensley M, Sonoda Y, Barakat R R, Anderson S., “Tissue Microarray Immunohistochemical Expression of Estrogen, Progesterone, and Androgen Receptors in Uterine Leiomyomata and Leiomyosarcoma”, Cancer 2004, 101(6):1455-62). On a functional level, androgens are known as strong growth drivers of the myometrium of the uterus in rat experiments (M. Gonzalez-Diddi, B. Komisaruk, and C. Beyer, “Differential Effects of Testosterone and Dihydrotestosterone on the Diverse Uterine Tissues of the Ovariectomized Rat”, Endocrinology 1972, 91(4): 1129f), and the expression of the androgen receptor in the myometrium and in leiomyoma is stimulated by estradiol. Furthermore, in situ synthesis in human myometrium and leiomyoma of the AR binding and transactivating androgens Testosterone and 5-alpha-Dehydro-testosterone has been shown in tissue culture experiments (V M Jasonni, M Bonavia, S Lodi, S Preti, C Bulletti, and C Flamigni, “Androstenedione metabolism in human uterine tissues: endometrium, myometrium and leiomyoma”, J Steroid Biochem. 1982, 17(5):547f). However, the effect of androgens and anti-androgens on the growth of uterine leiomyoma has not been evaluated so far.

Likewise, the effect of androgens and in particular of androgen receptor antagonists on proliferation of primary human myoma cells or Eker rat leimomyoma-tumor derived cells (Elt3) was not shown.

### DETAILED DESCRIPTION OF THE INVENTION

The aim of the present invention is to provide a new and effective treatment for curing and/or preventing fibroids, being also known as uterine leiomyoma, leiomyomata, or simply fibroids or myoma in mammals, preferably humans.

This is achieved by means of androgen receptor antagonists used for curing and/or preventing fibroids.

Particularly, said androgen receptor antagonists according to the invention, are non-steroidal androgen receptor antagonists which are used for curing and/or preventing fibroids in mammals, preferably humans.

According to an other form of embodiment of the invention said androgen receptor antagonists are steroidal androgen receptor antagonists which are used for curing and/or preventing fibroids in mammals, preferably humans

An androgen receptor antagonist, according to the invention, is chosen for example from the non-limiting list of: cyproterone acetate, oxendolone, chlormadinone acetate, spironolactone, osaterone acetate, dienogest, flutamide, hydroxyflutamide, nilutamide, bicalutamide, RU 58841, LGD-2226, MDV3100, BMS-641988, BMS-779333, or 4-(3-{[6-(2-hydroxy-2-methylpropoxy)pyridin-3-yl]methyl}-4,4-dimethyl-5-oxo-2-thioxoimidazolidin-1-yl)-2-(trifluoromethyl)benzonitrile (thioxoimidazolidine derivative) used for curing and/or preventing fibroids in mammals, preferably humans.

The invention further comprises the use of an androgen receptor antagonist in the manufacture of a medicament for curing and/or preventing fibroids.

According to a particular aspect of the invention androgen receptor antagonists, and more specifically steroidal androgen receptor antagonists, are to be considered with the proviso that said antagonists do not comprise dienogest and spironolactone, or more particularly dienogest, spironolactone, and cyproterone acetate.

Steroidal androgen receptor antagonists according to the present invention are compounds comprising in their chemical structure the following 4-rings system:

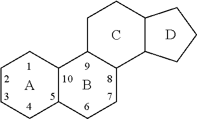

Said ring system is also known in the art as “steroidal skeleton”.

Furthermore, within the meaning of the present invention, androgen receptor antagonists refers also to selective androgen receptor modulators (SARMs). The structural basis of said SARMs might be either steroidal or non-steroidal.

The activity of steroidal SARMs in the treated mammal might, by purpose, also affect other steroid receptors such as the progesterone receptor and/or mineralocorticoid receptor.

The amount of an androgen receptor antagonist that is to be administered varies within a wide range and can cover any effective amount. On the basis of the condition that is to be treated and the type of administration, the amount of the compound that is administered can be 0.01 μg/kg-100 mg/kg of body weight, preferably 0.04 μg/kg-1 mg/kg of body weight, per day.

In humans, this corresponds to a dose of 0.8 μg to 8 g, preferably 3.2 μg to 80 mg, daily. According to the invention, a dosage unit considered for example as the single tablet, capsule, patch, suppository, ring, IUD contains etc. comprises 1.6 μg to 2000 mg of an androgen receptor antagonist.

The androgen receptor antagonists to be used according to the invention are suitable for the production of pharmaceutical compositions and preparations. The pharmaceutical compositions or pharmaceutical agents contain as active ingredients one or more of the androgen receptor antagonists, optionally mixed with other pharmacologically or pharmaceutically active substances. The production of the pharmaceutical agents is carried out in a known way, whereby the known and commonly used pharmaceutical adjuvants as well as other commonly used vehicles and diluents can be used.

As such vehicles and adjuvants, for example, those are suitable that are recommended or indicated in the following bibliographic references as adjuvants for pharmaceutics, cosmetics and related fields: Ullmans Encyklopädie der technischen Chemie [Ullman's Encyclopedia of Technical Chemistry], Volume 4 (1953), pages 1 to 39; Journal of Pharmaceutical Sciences, Volume 52 (1963), page 918 ff., issued by Czetsch-Lindenwald, Hilfsstoffe für Pharmazie and angrenzende Gebiete [Adjuvants for Pharmaceutics and Related Fields]; Pharm. Ind., Issue 2, 1961, p. 72 and ff.: Dr. H. P. Fiedler, Lexikon der Hilfsstoffe für Pharmazie, Kosmetik and angrenzende Gebiete [Dictionary of Adjuvants for Pharmaceutics, Cosmetics and Related Fields], Cantor K G, Aulendorf in Württemberg 1971.

The androgen receptor antagonist for the use according to the invention can be administered orally or parenterally, for example intraperitoneally, intramuscularly, subcutaneously or percutaneously. The androgen receptor antagonist can also be comprised in a dosage unit to be implanted in the tissue.

For oral administration, capsules, pills, tablets, coated tablets, etc., are suitable. In addition to the active ingredient, the dosage units can contain a pharmaceutically compatible vehicle, such as, for example, starch, sugar, sorbitol, gelatin, lubricant, silicic acid, talc, etc.

For parenteral administration, the active ingredients can be dissolved or suspended in a physiologically compatible diluent. As diluents, very often oils with or without the addition of a solubilizer, a surfactant, a suspending agent or an emulsifying agent are used. Examples of oils that are used are olive oil, peanut oil, cottonseed oil, soybean oil, castor oil and sesame oil.

The androgen receptor antagonist can also be used in the form of a depot injection or an implant preparation, which can be formulated so that a delayed release of active ingredient is made possible.

As inert materials, implants can contain, for example, biodegradable polymers, or synthetic silicones such as, for example, silicone rubber.

In addition, for percutaneous administration, the active ingredients can be added to, for example, a patch.

For the production of intravaginal systems (e.g., vaginal rings) or intrauterine systems (e.g., pessaries, coils, IUDs, Mirena®) that are loaded with an androgen receptor antagonist for local administration, various polymers are suitable, such as, for example, silicone polymers, ethylene vinyl acetate, polyethylene or polypropylene.

An androgen receptor antagonist for the use according of this invention may be administered rectally or vaginally in the form of a conventional suppository. Suppository formulations may be made from traditional materials, including cocoa butter, with or without the addition of waxes to alter the suppository's melting point, and glycerine. Water soluble suppository bases, such as polyethylene glycols of various molecular weights, may also be used.

According to the invention, the compounds of general formula I can also be encapsulated with liposomes.

The action of androgen receptor antagonists on the development of human fibroids was tested on a xenograft mouse model.

Using said disease model of human uterine leiomyoma xenografts from at least two different patients growing in immunodeficient mice, as described in more details below, it could be demonstrated that graft weights were significantly reduced by >50% in the thioxoimidazolidine derivative treatment group (p<0.05) when compared with controls. This unexpected effect demonstrates the role of anti-androgens in the treatment for fibroids, also known as uterine leiomyoma.

### BRIEF DESCRIPTION OF THE DRAWINGS

Other features and advantages of the invention will become evident on reading the following description of the reported embodiments of the invention, given as non-binding examples, to which enclosed drawings refer:

FIG. 1: Effect of different concentrations of testosterone on proliferation of Eker rat leiomyoma-derived cells (Elt3), DMSO=dimethyl sulfoxide, T=testosterone.

FIG. 2: Effect of different concentrations of dihydrotestosterone on proliferation of Eker rat leiomyoma-derived cells (Elt3), DMSO=dimethyl sulfoxide, DHT=5α-dihydrotestosterone.

FIG. 3: Effect of 10⁻⁶ M estradiol, testosterone and dihydrotestosterone on proliferation of primary myoma cells (*p<0.05 Two tailed unpaired t-Test vs. DMSO)

FIG. 4: Effect of 10⁻⁷ M bicalutamide as androgen receptor antagonist, on DHT-induced proliferation of Elt3 cells (*p<0.05 Two tailed unpaired t-Test vs. DMSO and vs. DHT+bic) DMSO=dimethyl sulfoxide, DHT=5α-dihydrotestosterone, T=testosterone, Bic=bicalutamide

FIG. 5: Graft weights for a test with bicalutamide as androgen receptor antagonist in an immunodeficient Xenograft mice model of human uterine leiomyoma (Experiment 1—see table below)

FIG. 6: Graft weights for a test with the thioxoimidazolidine derivative as androgen receptor antagonist in a immunodeficient Xenograft mice model of human uterine leiomyoma (Experiment 2—see table below)

FIG. 7: Human xenograft cell proliferation during the last week of Experiment 2 (see table below). Proliferation of grafts in mice treated with thioxoimidazolidine derivative is clearly reduced.

### SYNTHESIS OF THE NAMED ANDROGEN RECEPTOR ANTAGONISTS

The compounds cyproterone acetate, oxendolone, chlormadinone acetate, spironolactone, osaterone acetate, dienogest, flutamide, hydroxyflutamide, nilutamide, bicalutamide and their syntheses are well known in the pharmaceutical field, particularly in the field of substances active as androgen receptor antagonists. Compounds XI-XV can be prepared as described in prior art.

RU 58841 and its synthesis were described in WO1997/18197 (page 27, Example 4), LGD-2226 and its synthesis were described in WO2001/16108 (page 92, Example 21, Compound 223), MDV-3100 and its synthesis were described in WO2006/124118 (page 77, Example 56 [RD162′]), BMS-641988 and its synthesis were described in WO2003/062241 (A1) (page 618, Example 810), BMS-779333 and its synthesis were described in WO2009/003077 (A1) (page 70, Example 3).

The patent documents WO1997/18197, WO2001/16108, WO2006/124118, WO2003/062241 and WO 2009/003077 are incorporated herein in full by reference.

### 4-(3-{[6-(2-Hydroxy-2-methylpropoxy)pyridin-3-yl]methyl}-4,4-dimethyl-5-oxo-2-thioxoimidazolidin-1-yl)-2-(trifluoromethyl)benzonitrile (Thioxoimidazolidine Derivative—Example 10 of EP Patent Application 09075421.9)

2a) Production of intermediates

### Intermediate 2.1: 6-(2-Hydroxy-2-methylpropoxy)pyridine-3-carbonitrile

Sodium hydride (60%; 346 mg) was added to a solution of 2-methylpropane-1,2-diol (650 mg; 7.2 mmol) in N,N-dimethylformamide (66.7 ml) and the batch was stirred for 1 hour at room temperature. A solution of 6-chloropyridine-3-carbonitrile (1000 mg) in N,N-dimethylformamide (6.7 ml) was added and the batch was stirred over night at room temperature. The mixture was diluted with ice and a diluted solution of sodium chloride and extracted with ethyl acetate (3×). The combined organic phases were washed with a diluted sodium chloride solution, dried over sodium sulfate and filtered. The filtrate was concentrated in vacuo and the residue was purified by column chromatography (hexane→hexane/ethyl acetate 1:1) to give the desired product (508 mg; 2.6 mmol).

¹H-NMR (300 MHz, CDCl₃): 8.46 (d, 1H), 7.81 (dd, 1H), 6.88 (d, 1H), 4.26 (s, 2H), 2.35 (br, 1H), 1.33 (s, 6H).

### Intermediate 2.2: 6-(2-Hydroxy-2-methylpropoxy)pyridine-3-methanamine

A solution of 6-(2-hydroxy-2-methylpropoxy)pyridine-3-carbonitrile (400 mg; 2.08 mmol) in a 7 N solution of ammonia in methanol (20 ml) was hydrogenated in an autoclave at 25° C. with the use of Raney Nickel (400 mg; 50%) under a hydrogen atmosphere of 20 bar for 5 hours. The batch was filtered and concentrated by evaporation to yield the crude product (415 mg) that was used without further purification.

2b) Production of title compound

6-(2-Hydroxy-2-methylpropoxy)pyridine-3-methanamine (408 mg; 2.08 mmol) was suspended in tetrahydrofuran (10 ml). After the addition of acetone cyanohydrin (0.38 ml; 4.16 mmol, Fluka), and molecular sieves (4 Å) the reaction was stirred over night at room temperature. The reaction was filtered and concentrated by evaporation.

The residue was taken up in tetrahydrofuran (9 ml). 4-Isothiocyanato-2-(trifluoromethyl)benzonitrile (432 mg; 1.89 mmol, Fluorochem) and triethylamine (0.53 ml; 3.78 mmol) were added and the reaction was refluxed for 1 hour before it was concentrated by evaporation.

The residue was taken up in methanol (5.7 ml). A 4 N solution of hydrogen chloride in methanol (1.89 ml) was added and the reaction was stirred over night at room temperature. The reaction was diluted with ethyl acetate and washed with saturated solutions of sodium bicarbonate and sodium chloride. The organic phase was filtered using a Whatman filter and concentrated by evaporation. The residue was purified by column chromatography (dichloromethane/ethanol 95:5) to yield the title compound (165 mg; 0.34 mmol).

¹H-NMR (300 MHz, CDCl₃): 8.17 (d, 1H), 7.97 (d, 1H), 7.91 (m, 1H), 7.82 (dd, 1H), 7.79 (dd, 1H), 6.82 (d, 1H), 5.05 (s, 2H), 4.21 (s, 2H), 3.08 (br, 1H), 1.50 (s, 6H), 1.33 (s, 6H).

### ASSESSMENT OF ANDROGEN RECEPTOR ANTAGONISTS ON BIOLOGICAL MODELS

Effects of Two Different Androgens on Eker Rat Leiomyoma Tumor-Derived Cells (Elt3)

Elt3 cells were obtained from Cheryl Walker (University of Texas). In comparison to other cells, Elt3 cells were characterized to be tumorigenic in nude mice (C. Walker, Recent Prog. Horm. Res., January 2002; 57: 277). Elt3 cells were grown to confluence in T-75 flasks with DMEM containing 10% FCS at 37° C. and 5% CO2. The medium was changed into 1% CCS for 24 h. 1000 cells were seeded in 96-well-plates for 24 hours. Elt3 cells were incubated with test compounds for 7 days. The CellTiter-Glo® Luminescent Cell Viability Assay (Promega) was used for determination of the number of viable cells in culture based on quantitation of the ATP present, an indicator of metabolically active cells.

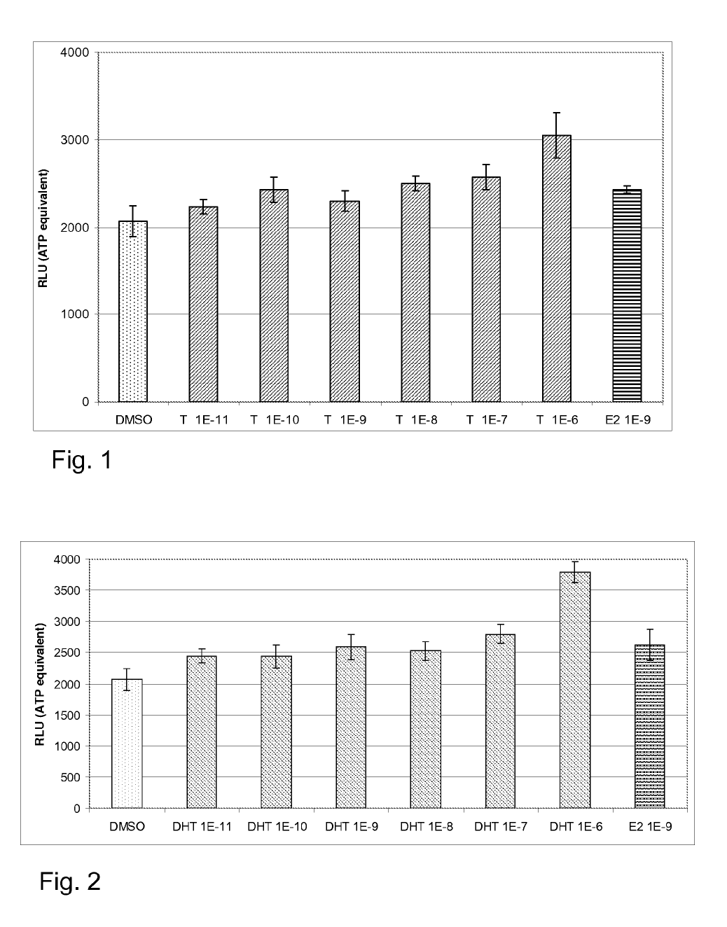

As it clearly appears in FIG. 1 and FIG. 2 respectively, it was shown that testosterone and dihydrotestosterone induce dose-dependent increase of proliferation of Eker rat myoma cell line (Elt3) in a range comparable to estradiol.

Isolation of Primary Myoma and Myometrial Cells from Human Tissue

Human uterine leiomyoma and matched myometrial tissues were obtained at surgery from non-pregnant women undergoing hysterectomy for medically indicated reasons. Myometrial and leiomyoma tissues were dissected from endometrial cell layers, washed in PBS to remove blood cells, cut into small pieces of about 1 mm³ and were digested in 2% collagenase II and 0.1% DNase I over night at 4° C. The cells were separated from undigested pieces by centrifugation at 295×g for 15 min, washed twice with DMEM containing 10% FCS and 1% antibiotic/antimycotic solutions. For removal of most of the ‘unwanted’ fibroblast, the cells were incubated in T25 plastic flasks for one hour. The supernatant containing the myometrial/myoma cells was incubated for three to four days with DMEM containing 10% FCS and 1% antibiotic/antimycotic solutions at 37° C. and 5% CO₂. These primary cells (passage 2-4) were used for further experiments to evaluate the proliferative effects of different compounds.

Effects of Estradiol and Two Different Androgens on Primary Myoma Cells

The primary cells were grown to confluence in T-75 flasks with DMEM containing 10% FCS at 37° C. and 5% CO₂. 1000 cells were seeded in 96-well-plates for 24 hours and the medium was changed into 1% CCS. The primary cells were incubated with different test compounds for 7 days. The CellTiter-Glo® Luminescent Cell Viability Assay (Promega) was used for determination of the number of viable cells in culture based on quantitation of the ATP present, an indicator of metabolically active cells.

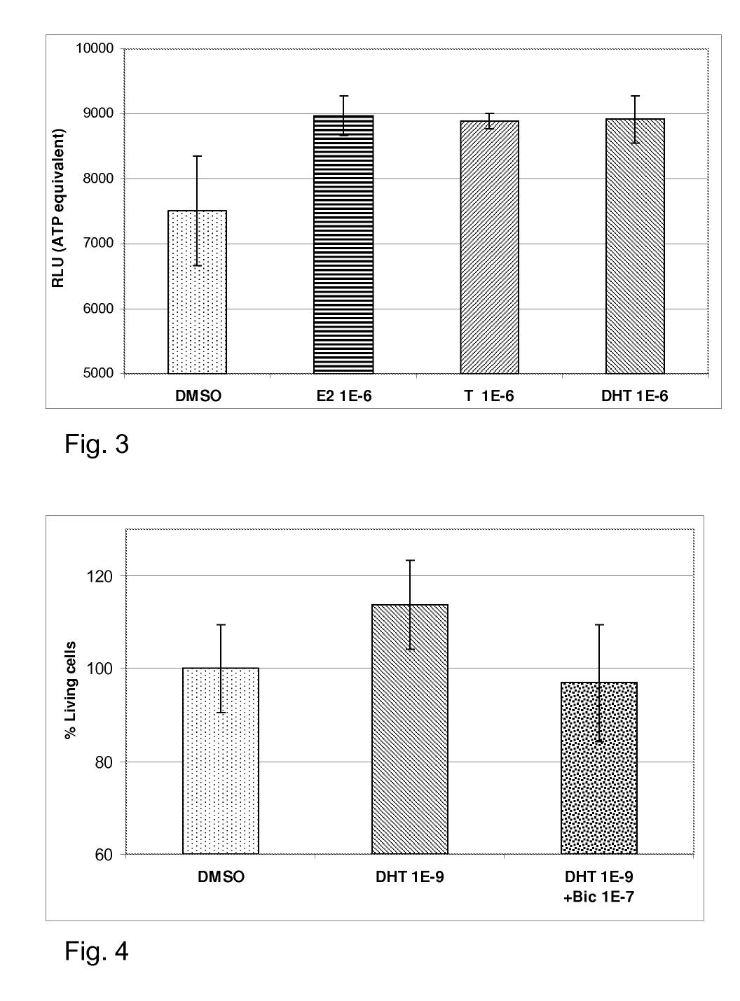

It was shown (FIG. 3) that dihydrotestosterone and testosterone significantly induce an increase of proliferation of primary myoma cells in a range comparable to estradiol (p<0.05 Two tailed unpaired t-Test vs. DMSO).

Effects of Bicalutamide on Eker Rat Leiomyoma Tumor-Derived Cells (Elt3)

Elt3 cells were obtained and grown to confluence and were seeded as described above. Elt3 cells were incubated with test compounds for 7 days. The CellTiter-Glo® Luminescent Cell Viability Assay (Promega) was used for determination of the number of viable cells in culture based on quantitation of the ATP present, an indicator of metabolically active cells.

According to the results summarized in FIG. 4, the proliferation of Elt3 cells was significantly increased by 10⁻⁹ M DHT (p<0.05 Two tailed unpaired t-Test vs. DMSO) and this effect was abolished by 10⁻⁷ M bicalutamide (p<0.05 Two tailed unpaired t-Test vs. DHT).

Xenograft of Human Uterine Leiomyoma in Immunodeficient Mice

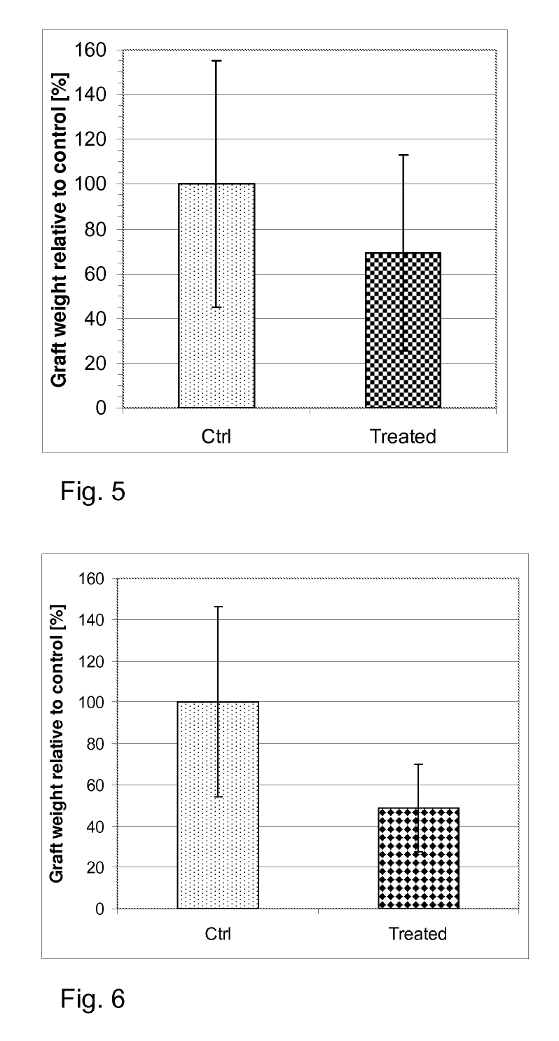

Human uterine leiomyoma (UL) tissue was derived from any form of surgical intervention indicated for the respective diagnosis, either by hysterectomy with subsequent preparation of the tissues or by myomenucleation with or without morcellation for removal of the tissue from the abdominal cavity. Immediately after the surgery the UL tissue was placed into an appropriate sterile buffer (Vitron V7 buffer (U.S. Pat. No. 5,328,821) or Viaspan buffer for organ transplantation) at 4° C. for transport from the clinic. Under a sterile workbench, the UL tissue was cut into small pieces of 2×2×2 mm while keeping the tissue constantly moist. The small pieces of tissue were placed in tissue wells with PBS at room temperature for xenotransplantation. (M Fritsch et al. 2010, ISGE abstract & presentation). Immunodeficient mice (CB17 SCID, ICR SCID, ICR-Hrhr SCID or SCID beige mice) were ovarectomized (OVX) at an age of approx. 6-8 weeks. At least one week after OVX, the mice were supplemented with estradiol (E2) (0.05 mg/90 d) and progesterone (P) (25 mg/60 d) releasing pellets (Innovative Research of America) in the neck area. Up to eight grafts with tissue from one donor were placed subcutaneously in the ventral area, either four UL and four myometrial tissue grafts as a control, or eight UL grafts. The surgical cuts were closed with clips or sealed with tissue glue (Histoacryl, Braun). Immediately after surgery, the mice were divided into two groups. One group received vehicle by gavage. The other group received an anti-androgen (thioxoimidazolidine derivative or bicalutamide) by gavage in the same vehicle. Application was either daily or every other day; depending on the biological half life of the compound. For the last week of the experiment, the mice received bromodeoxyuridine (BrdU) in their drinking water for subsequent analysis of graft cell proliferation. After a given time of treatment (50 d or 60 d), the experiment was stopped and the grafts were prepared. UL tissue is characterized by excessive synthesis of extracellular matrix and by enhanced growth rate as compared to myometrium. The grafts had been shown to preserve typical characteristics of UL tissue while growing. Therefore, graft weight was taken as a primary read-out parameter for growth of UL tissue (FIGS. 5 and 6).

Experiment 1 (Bicalutamide as Androgen Receptor Antagonist)—FIG. 5
Human myoma tissue from three different patients was grafted s.c. in SCID mice; and the mice were treated as described in method below. Graft weights were normalized to the weight of the respective control group, and analyzed by the statistical method described.
As displayed in FIG. 5, graft weight was significantly reduced by >30% in the bicalutamide treatment group (p<0.05) when compared to the respective controls

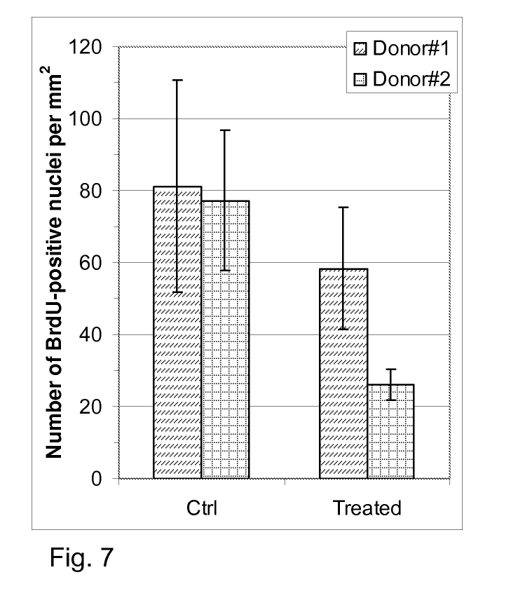

Experiment 2 (the Thioxoimidazolidine Derivative as Androgen Receptor Antagonist)—FIGS. 6 and 7
Human myoma tissue from two different patients was grafted s.c. in SCID mice; and the mice were treated as described in method above. The graft weights were normalized to the weight of the respective control group, and analyzed by the statistical method described.
As it clearly appears in the FIG. 6, graft weight was significantly reduced by >50% in the thioxoimidazolidine derivative treatment group (p<0.05) when compared to the respective controls.
Human xenograft cell proliferation during the last week of Experiment 2, for which the respective graft weights were shown in FIG. 6, was analyzed. BrdU positive nuclei were visualized by immunohistochemical staining in formalin-fixed graft sections, pictures were taken, and BrdU-positive nuclei per area were counted using the MIRAX Histoquant software (3DHISTECH Ltd, Budapest, Hungary). Graft growth and proliferation may vary with each individual myoma used for the grafting experiment. Therefore the proliferation data were shown separately for the two different donors of Experiment 2 (FIG. 7).
Proliferation of grafts in mice treated with thioxoimidazolidine derivative is clearly reduced as shown in FIG. 7.

It could be demonstrated that graft weights significantly (p<0.05) decreased by >30% for bicalutamide and >50% under treatment with the thioxoimidazolidine derivative when compared with matched grafts of the same patients in the respective control groups and an inhibition of the growth of the transplanted human fibroid tissue was achieved for both tested androgen receptor antagonists.

Experiment 1: Treatment Groups for Bicalutamide

| | | | Test | | |
|---|---|---|---|---|---|
| | | | com- | | |
| | | | pound | Treat- | |
| | | | Dose | ment | |
| | | | [mg/ | dura- | |
| group | Treatment | | kg/d] | tion | Sample size |
| 1 | E2 pellet | 0.1 mg/60 d | 0.022 | 60 d | 14 mice/72 grafts |
| | P pellet | 25 mg/60 d | 16.6 | | total |
| | Vehicle | p.o. | | 60 d | 4/32 from |
| | | | | | patient 1, |
| | 0.5% Tween80 in H₂O | | | | 5/20 from |
| | | | | | patient 2, |
| | | | | | 5/20 from |
| | | | | | patient 3 |
| 2 | E2-Pellet | 0.05 mg/90 d | 0.022 | 60 d | 15 mice/80 grafts |
| | P-Pellet | 25 mg/60 d | 16.6 | | total |
| | Bicalutamide | p.o. | 15 | 60 d | 5/40 from |
| | | | | | patient 1, |

| | |
|---|---|
| every 2^{nd} day in vehicle | 5/20 from |
| | patient 2, |
| | 5/20 from |
| | patient 3 |

Experiment 2: Treatment Groups for the Thioxoimidazolidine Derivative

| | | | Test | | |
|---|---|---|---|---|---|
| | | | com- | | |
| | | | pound | Treat- | |
| | | | Dose | ment | |
| | | | [mg/ | dura- | |
| group | Treatment | | kg/d] | tion | Sample size |
| 1 | E2 pellet | 0.1 mg/60 d | 0.022 | 60 d | 9 mice/72 grafts |
| | | | | | total |
| | P pellet | 25 mg/60 d | 16.6 | 60 d | 5/40 from patient 4, |
| | Vehicle | p.o. | | | 4/32 from patient 5 |
| | NMP + PEG300 1 + 9 | | | | |
| 2 | E2-Pellet | 0.05 mg/90 d | 0.022 | 60 d | 8 mice/64 grafts |
| | | | | | total |
| | P-Pellet | 25 mg/60 d | 16.6 | 60 d | 4/32 from patient 4, |

| | | |
|---|---|---|
| thioxoimidazolidine | 15 | 4/32 from patient 5 |
| derivative p.o. in | | |
| vehicle daily | | |

P = progesterone E2 = estradiol

Statistical Analysis of the Experiment

A lognormal distribution for the observed graft weights was assumed. A mixed linear model was applied to the log of the graft weights using “treatment” as fixed and “tissue” as a random effect. In order to describe the correlation between measurements per mouse a compound symmetry structure was used. Degrees of freedom were adjusted according to Satterthwaite. All treatment groups were compared with the positive control group using one-sided Dunnett's tests.

1-8. (canceled)

9. A method of treating or preventing fibroids in a human comprising the step of administering an androgen receptor antagonist with the proviso that said androgen receptor antagonist is not dienogest or spironolactone.

10. The method of claim 9, wherein the androgen receptor antagonist is a steroidal androgen receptor antagonist.

11. The method of claim 9, wherein the androgen receptor antagonist is a non-steroidal androgen receptor antagonist.

12. The method of claim 9, wherein the androgen receptor antagonist is cyproterone acetate, oxendolone, chlormadinone acetate, osaterone acetate, flutamide, hydroxyflutamide, nilutamide, bicalutamide, RU 58841, LGD-2226, MDV3100, BMS-641988, BMS-779333, or 4-(3-{[6-(2-hydroxy-2-methylpropoxy)pyridin-3-yl]methyl}-4,4-dimethyl-5-oxo-2-thioxoimidazolidin-1-yl)-2-(trifluoromethyl)benzonitrile.

13. The method of claim 9, wherein the androgen receptor antagonist is administered orally.

14. The method of claim 9, wherein the androgen receptor antagonist is administered by a transdermal patch.

15. The method of claim 9, wherein the androgen receptor antagonist is administered by an intravaginal system.

16. The method of claim 9, wherein the androgen receptor antagonist is administered by an intrauterine system.
